(19) United States
(12) Patent Application Publication
(10) Pub. No.: US 2026/0174824 A1
(43) Pub. Date: Jun. 25, 2026

(54) ANTIBIOTIC-FREE PLASMID MAINTENANCE SYSTEMS AND METHODS OF USING SAME

(71) Applicant: Wisconsin Alumni Research Foundation, Madison, WI (US)

(72) Inventors: Jan Peter van Pijkeren, Madison, WI (US); Zhiying Wang, Madison, WI (US); Jeehwan Oh, Madison, WI (US)

(73) Assignee: Wisconsin Alumni Research Foundation, Madison, WI (US)

(21) Appl. No.: 19/351,511

(22) Filed: Oct. 7, 2025

Related U.S. Application Data
(60) Provisional application No. 63/704,193, filed on Oct. 7, 2024.

(51) Int. Cl. A61K 38/00; C12N 9/10; C12N 15/52

(52) U.S. Cl. CPC A61K 38/00; C12N 9/1029; C12N 15/52

(57) ABSTRACT

Antibiotic-free systems for maintaining plasmids in host cells and methods of using same. The plasmids can comprise one or more plasmid-maintenance genes and a gene of interest configured to express in the host cell. The one or more plasmid-maintenance genes can comprise one or more of a prophage repressor gene, a sucrose phosphorylase gene, and an alcohol/aldehyde dehydrogenase gene. The methods can comprise growing the host cell with the plasmid comprised within the host cell, wherein the plasmid can be maintained during growth in the host cell without the use of antibiotics. In systems with a plasmid comprising a sucrose phosphorylase gene, the methods can comprise growing the host cell in the presence of sucrose as a sole carbon source. In systems with a plasmid comprising an alcohol/aldehyde dehydrogenase gene, the methods can comprise growing the host cell in the presence of glucose as a sole carbon source.

### CROSS-REFERENCE TO RELATED APPLICATIONS

Priority is hereby claimed to U.S. Provisional Application 63/704,193, filed Oct. 7, 2024, which is incorporated herein by reference in its entirety.

### STATEMENT REGARDING FEDERALLY SPONSORED RESEARCH

This invention was made with government support under AT011202 awarded by the National Institutes of Health. The government has certain rights in the invention.

### SEQUENCE LISTING

The instant application contains a Sequence Listing which has been submitted in XML format and is hereby incorporated by reference in its entirety. The XML copy, created on Sep. 12, 2025, is named USPTO-250912-09824637-P240157US02-SEQ_LIST.xml and is 20,489 bytes in size.

### FIELD OF THE INVENTION

The invention is directed to antibiotic-free systems for maintaining plasmids in host cells and methods of using same.

### BACKGROUND

Plasmids include small, circular DNA molecules that are separate from the bacterial chromosome. Plasmids are indispensable tools in microbiology research as they enable various applications, including, but not limited to, facilitating gene transfer between bacteria, assisting in genetic engineering, aiding in vaccine development, and finding applications in bioremediation and biotechnology (Kroll et al. 2010). To maintain recombinant plasmids in the cell, genes encoding antibiotic resistance are typically employed (Hall et al. 2004). However, the presence of antibiotic resistance genes is undesirable because it poses a potential risk of horizontal transfer of antibiotic resistance genes to other microbes in the community (Bennett et al. 2008). This transfer can contribute to the spread of antibiotic resistance, making it an important focus of research and public health efforts (Huddleston et al. 2014). Besides safety risk, antibiotic resistance genes in the plasmids are associated with structural plasmid instabilities and decreased gene delivery efficiency (Mairhofer et al. 2008). These drawbacks have highlighted the need for developing alternative approaches that do not rely on antibiotic selection when working with plasmids. Antibiotic-free systems for maintaining plasmids in host cells are needed.

### SUMMARY OF THE INVENTION

One aspect of the invention is directed to systems. The systems can comprise a microbial host cell and a recombinant plasmid capable of replicating in the host cell. The plasmid can comprise one or more plasmid-maintenance genes configured to express in the host cell and a gene of interest configured to express in the host cell. The one or more plasmid-maintenance genes can comprise one or more of a prophage repressor gene, a sucrose phosphorylase gene, and an alcohol/aldehyde dehydrogenase gene.

In some versions, the one or more plasmid-maintenance genes comprise a prophage repressor gene. In some versions, the host cell comprises a genome comprising a prophage. In some versions, the prophage repressor gene encodes a repressor of the prophage. In some versions, the prophage repressor gene encodes a protein comprising a sequence at least 80% identical to a sequence selected from the group consisting of SEQ ID NOS:2 and 4.

In some versions, the one or more plasmid maintenance genes comprises a sucrose phosphorylase gene. In some versions, the sucrose phosphorylase gene encodes a protein comprising a sequence at least 80% identical to SEQ ID NO:6. In some versions, the host cell comprises a mutation that reduces sucrose phosphorylase activity with respect to a native form of the host cell. In some versions, the host cell comprises an inactivating mutation to a native sucrose phosphorylase gene present in a native form of the host cell. In some versions, the native sucrose phosphorylase gene encodes a protein comprising a sequence at least 80% identical to SEQ ID NO:6.

In some versions, the one or more plasmid maintenance genes comprises an alcohol/aldehyde dehydrogenase gene. In some versions, the alcohol/aldehyde dehydrogenase gene encodes a protein comprising a sequence at least 80% identical to SEQ ID NO:8. In some versions, the host cell comprises a mutation that reduces alcohol/aldehyde dehydrogenase activity with respect to a native form of the host cell. In some versions, the host cell comprises an inactivating mutation to a native alcohol/aldehyde dehydrogenase gene present in a native form of the host cell. In some versions, the native sucrose phosphorylase gene encodes a protein comprising a sequence at least 80% identical to SEQ ID NO:8.

In some versions, the plasmid is devoid of any antibiotic resistance gene.

In some versions, the host cell is a bacterium. In some versions, the host cell is a member of Limosilactobacillus. In some versions, the host cell is Limosilactobacillus reuteri.

Another aspect of the invention is directed to methods of using the systems of the invention. In some versions, the methods comprise growing the host cell with the plasmid comprised within the host cell. In some versions, the methods comprise expressing the gene of interest.

In some versions, the one or more plasmid maintenance genes comprises a sucrose phosphorylase gene and the growing comprises growing the host cell in the presence of sucrose. In some versions, the growing comprises growing the host cell in the presence of sucrose as a sole carbon source.

In some versions, the one or more plasmid maintenance genes comprises an alcohol/aldehyde dehydrogenase gene and the growing comprises growing the host cell in the presence of glucose. In some versions, the growing comprises growing the host cell in the presence of glucose as a sole carbon source.

In some versions, the methods comprise administering the host cell to a subject and growing the host cell in vivo. In some versions, the methods comprise orally administering the host cell to a subject and growing the host cell in vivo in a gastrointestinal tract of the subject. In some versions, the one or more plasmid maintenance genes comprises a sucrose phosphorylase gene and the method further comprises administering sucrose to the gastrointestinal tract. In some versions, the one or more plasmid maintenance genes comprises an alcohol/aldehyde dehydrogenase gene and the method further comprises administering glucose to the gastrointestinal tract.

The objects and advantages of the invention will appear more fully from the following detailed description of the preferred embodiment of the invention made in conjunction with the accompanying drawings.

### BRIEF DESCRIPTION OF THE DRAWINGS

FIG. 1 Map of pSIP-eftu-R and the DNA sequence of the phage repressor.

FIG. 2. Comparison of the phage production of L. reuteri harboring pSIP-control and pSIP-eftu-R. The overnight culture was used to inoculate a fresh batch of MRS medium supplemented with erythromycin to reach an initial OD of 0.1. After 4-h incubation at 37° C., supernatants were collected and filter sterilized, followed by PFU analysis. Data were shown as Mean±SD based on three biological replicates. The significance level of p<0.05 is indicated by the asterisk symbol (*) in the results.

FIG. 3. The plasmid stability of pSIP-eftu-R in L. reuteri when cultured in antibiotic-free MRS after 200 generations. The cells of L. reuteri (pSIP-control) and L. reuteri (pSIP-eftu-R) were subcultured in fresh MRS every 24 h for 20 times. After the 200 generations, the cultures were subjected to serial dilution and plated on two types of agar plates: MRS agar plates (to determine total cell counts) and MRS agar plates supplemented with erythromycin (to determine the cell counts of cells harboring the plasmids). By comparing the cell counts, the plasmid lost could be assessed. Data were shown as Mean±SD based on two biological replicates.

FIG. 4A. The optical density at 600 nm (OD₆₀₀) of L. reuteri WT (WT), SucP mutant (ΔsucP), and ΔsucP harboring sucP expression plasmid (ΔsucP+pSucP) in the mMRS supplemented with no carbohydrate (Control), 100 mM glucose (Glu 100), or 50 mM sucrose (Suc 50). All bacterial cultures were grown at 37° C. for 24 h. FIG. 4B Viable cell number (CFU) of L. reuteri harboring control plasmid (pControl) and ΔsucP+pSucP after ten passages in Suc 50 without antibiotics. Both strains were plated on Suc 50 agar plates and grown at 37° C. for 24 h in hypoxic condition. The mMRS was manually prepared by adding all MRS ingredients except for sugars and sodium acetate. Data were shown as Mean±SD based on two biological replicates.

FIG. 5A. The optical density at 600 nm (OD₆₀₀) of L. reuteri WT (WT), AdhE mutant (ΔadhE), and ΔadhE harboring adhE expression plasmid (ΔadhE+pAdhE) in the mMRS supplemented with no carbohydrate (Control), 100 mM glucose (Glu 100), or 50 mM sucrose (Suc 50). All bacterial cultures were grown at 37° C. for 24 h. FIG. 5B. Viable cell number (CFU) of L. reuteri harboring control plasmid (pControl) and ΔadhE+pAdhE after ten passages in Glu 100 without antibiotics. Both strains were plated on Glu 100 agar plates and grown at 37° C. for 24 h in hypoxic condition. The mMRS was manually prepared by adding all MRS ingredients except for sugars and sodium acetate. Data were shown as Mean±SD based on two biological replicates.

### DETAILED DESCRIPTION OF THE INVENTION

The systems of the invention can comprise a microbial host cell and a plasmid. The plasmid can be a recombinant plasmid.

“Plasmid” as used herein refers to an extrachromosomal nucleic molecule that can replicate independently from the chromosomal DNA within a cell. Plasmids are typically capable of replicating independently from chromosomal DNA by virtue of an origin of replication. The plasmids of the invention are preferably configured to replicate in the host cell, whether by virtue of a particular origin of replication or by virtue of another element and/or mechanism. The plasmids of the invention can range in size from very small mini-plasmids of less than 1-kilobases (kb) to very large megaplasmids of several megabases (Mb). The plasmids of the invention can be circular or linear. The plasmids of the invention can be double stranded or single stranded. The plasmids of the invention can comprise DNA or RNA. The plasmids of the invention can be high copy number, medium copy number, low copy number, or very low copy number. In some versions of the invention, the plasmid is a circular, double-stranded DNA molecule having size of at least 0.5 kb, at least 0.75 kb, at least 1 kb and/or up to 200 kb, 300 kb, 400 kb, 500 kb, 600 kb, 700 kb, 800 kb, 900 kb, 1 Mb, or more.

The microbial host cell can comprise a cell of any type of microbe. The microbial host can be a recombinant microbial host.

The host cells of the invention can comprise bacteria. Bacteria of the invention can include certain commensal or probiotic bacteria, non-commensal bacteria, and other types of bacteria. The bacteria can include non-pathogenic, Gram-positive bacteria capable of anaerobic growth. The bacteria in some cases are viable in the gastrointestinal tract of mammals. The bacteria can be food grade. Other exemplary bacteria of the invention include E. coli.

Exemplary bacteria of the invention include species of lactic acid bacteria (i.e., species of the order Lactobacillales), such as those from the genera Lactobacillus, Limosilactobacillus, Leuconostoc, Pediococcus, Lactococcus, Streptococcus, Aerococcus, Carnobacterium, Enterococcus, Oenococcus, Fructobacillus, Sporolactobacillus, Tetragenococcus, Vagococcus, and Weissella.

Exemplary bacteria include species of the Lactobacillus genus. Exemplary species from the Lactobacillus genus include L. acetototerans, L. acidifarinae, L. acidipiscis, L. acidophilus, L. agilis, L. algidus, L. atimentarius, L. amytolyticus, L. amylophilus, L. amylotrophicus, L. amylovorus, L. animatis, L. antri, L. apodemi, L. aviarius, L. bifermentans, L. brevis, L. buchneri, L. camelliae, L. casei, L. catenaformis, L. ceti, L. coleohominis, L. collinoides, L. composti, L. concavus, L. coryniformis, L. crispatus, L. crustorum, L. curvatus, L. delbrueckii subsp. delbrueckii, L. delbrueckii subsp. butgaricus, L. delbrueckii subsp. lactis, L. dextrinicus, L. diolivorans, L. equi, L. equigenerosi, L. farraginis, L. farciminis, L. fermentum, L. fornicalis, L. fructivorans, L. frumenti, L. fuchuensis, L. gallinarum, L. gasseri, L. gastricus, L. ghanensis, L. graminis, L. hammesii, L. hamsteri, L. harbinensis, L. hayakitensis, L. helveticus, L. hitgardii, L. homohiochii, L. iners, L. ingluviei, L. intestinalis, L. jensenii, L. johnsonii, L. katixensis, L. kefiranofaciens, L. kefiri, L. kimchii, L. kitasatonis, L. kunkeei, L. leichmannii, L. lindneri, L. malefermentans, L. mati, L. manihotivorans, L. mindensis, L. mucosae, L. murinus, L. nagelii, L. namurensis, L. nantensis, L. oligofermentans, L. oris, L. panis, L. pantheris, L. parabrevis, L. parabuchneri, L. paracollinoides, L. parafarraginis, L. parakefiri, L. paratimentarius, L. paraplantarum, L. pentosus, L. perolens, L. plantarum, L. pontis, L. psittaci, L. rennini, L. reuteri, L. rhamnosus, L. rimae, L. rogosae, L. rossiae, L. ruminis, L. saerimneri, L. sakei, L. salivarius, L. sanfranciscensis, L. satsumensis, L. secaliphilus, L. sharpeae, L. siliginis, L. spicheri, L. suebicus, L. thailandensis, L. ultunensis, L. vaccinostercus, L. vaginalis, L. versmoldensis, L. vini, L. vitulinus, L. zeae, and L. zymae.

Exemplary bacteria include species of the Limosilactobacillus genus. Exemplary species from the Limosilactobacillus genus include L. agrestis, L. albertensis, L. alvi, L. antri, L. balticus, L. caviae, L. coleohominis, L. equigenerosi, L. fastidiosus, L. fermentum, L. frumenti, L. gastricus, L. gorilla, L. ingluviei, L. mucosae, L. oris, L. panis, L. pontis, L. portuensis, L. reuteri, L. rudii, L. secaliphilus, L. urinaemulieris, and L. vaginalis.

Exemplary bacteria of the invention include species of Bifidobacterium. Exemplary species from the Bifidobacterium genus include B. actinocoloniiforme, B. adolescentis, B. aemilianum, B. aerophilum, B. aesculapii, B. amazonense, B. angulatum, B. animalis, B. anseris, B. apousia, B. apri, B. aquikefiri, B. asteroides, B. avesanii, B. biavatii, B. bifidum, B. bohemicum, B. bombi, B. boum, B. breve, B. callimiconis, B. callitrichidarum, B. callitrichos, B. canis, B. castoris, B. catenulatum, B. catulorum, B. cebidarum, B. choerinum, B. choladohabitans, B. choloepi, B. colobi, B. commune, B. criceti, B. crudilactis, B. cuniculi, B. dentium, B. dolichotidis, B. eriksonii, B. erythrocebi, B. eulemuris, B. faecale, B. felsineum, B. gallicum, B. gallinarum, B. globosum, B. goeldii, B. hapali, B. indicum, B. italicum, B. jacchi, B. lemurum, B. leontopitheci, B. longum, B. magnum, B. margollesii, B. merycicum, B. miconis, B. miconisargentati, B. minimum, B. mongoliense, B. moraviense, B. moukalabense, B. myosotis, B. oedipodis, B. olomucense, B. panos, B. parmae, B. platyrrhinorum, B. pluvialisilvae, B. polysaccharolyticum, B. pongonis, B. porcinum, B. primatium, B. pseudocatenulatum, B. pseudolongum, B. psychraerophilum, B. pullorum, B. ramosum, B. reuteri, B. rousetti, B. ruminale, B. ruminantium, B. saguini, B. saguinibicoloris, B. saimiriisciurei, B. samirii, B. santillanense, B. scaligerum, B. scardovii, B. simiarum, B. simiiventris, B. stellenboschense, B. subtile, B. thermacidophilum, B. thermophilum corrig., B. tibiigranuli, B. tissieri corrig., B. tsurumiense, B. urinalis, B. vansinderenii, B. vespertilionis, and B. xylocopae.

A bacterium used in the following examples is L. reuteri (Limosilactobacillus reuteri formerly referred to as Lactobacillus reuteri). In addition to L. reuteri, other particularly preferred bacteria include L. plantarum (e.g., L. plantarum BAA-793), L. rhamnosus (e.g., L. rhamnosus GG (L. rhamnosus ATCC 53103)), L. lactis (e.g., L. lactis MG1363), and L. casei.

The plasmids of the invention can comprise one or more plasmid-maintenance genes configured to express in the host cell. “Plasmid-maintenance gene” as used herein refers to a gene that can, under at least some conditions, enhance maintenance of the plasmid in a host cell. “Plasmid maintenance” (and grammatical variants thereof), also referred to in the art as “plasmid stability,” “segregational stability,” or plasmid persistence, refers to the maintained presence of a plasmid in a population of cells upon growth (cell division). Plasmid maintenance can be indicated, for example, by a percentage of cells that contain the plasmid. Plasmid maintenance is the opposite of plasmid loss, plasmid instability, and segregational instability. The plasmid-maintenance genes of the invention can be recombinant genes. The plasmid-maintenance genes of the invention can be configured to constitutively express in the host cell or inducibly express in the host cell.

The plasmid-maintenance gene can be a recombinant gene. The recombinant plasmid-maintenance gene can comprise a coding sequence of the biologic operably linked to a promoter. The promoter can be heterologous to the coding sequence. In some versions, the promoter is a constitutive promoter. In some versions, the promoter is an inducible promoter.

In some versions, the one or more plasmid-maintenance genes comprise a prophage repressor gene. Prophage repressor genes are also known as “phage repressor” genes. Prophage repressor genes are genes that repress lytic phage growth, typically by expressing repressor proteins that bind to specific operator sites in prophage genomes to repress the transcription of genes required for lytic growth. Prophage repressor genes are well known in the art. See, e.g., Canchaya et al. 2003 (Canchaya C, Proux C, Fournous G, Bruttin A, Brussow H. Prophage genomics. Microbiol Mol Biol Rev. 2003 June; 67(2):238-76. Erratum in: Microbiol Mol Biol Rev. 2003 September; 67(3):473), Aggarwal et al. 1988 (Aggarwal, A. K., Rodgers, D. W., Drottar, M., Ptashne, M., Harrison, S. C.: Recognition of a DNA operator by the repressor of phage 434: a view at high resolution. Science 242: 899-907 (1988)), Dodd et al. 2001 (Dodd I B, Perkins A J, Tsemitsidis D, Egan J B. Octamerization of lambda CI repressor is needed for effective repression of P(RM) and efficient switching from lysogeny. Genes Dev. 2001 Nov. 15; 15(22):3013-22), Daou et al. 2013 (Daou N, Yu C, McClure R, Gudino C, Reed G W, Genco C A. Neisseria prophage repressor implicated in gonococcal pathogenesis. Infect Immun. 2013 October; 81(10):3652-61), among others.

In some versions, the prophage repressor gene encodes a protein comprising a sequence at least 40% identical, at least 45% identical, at least 50% identical, at least 55% identical, at least 60% identical, at least 65% identical, at least 70% identical, at least 75% identical, at least 80% identical, at least 85% identical, at least 90% identical, at least 95% identical, at least 99% identical, or 100% identical to SEQ ID NO:2. An exemplary coding sequence of SEQ ID NO:2 is SEQ ID NO:1.

In some versions, the prophage repressor gene encodes a protein comprising a sequence at least 80% identical, at least 85% identical, at least 90% identical, at least 95% identical, at least 99% identical, or 100% identical to SEQ ID NO:4. In exemplary coding sequence of SEQ ID NO:4 is SEQ ID NO:3.

In some versions, the host cell in the system comprises a genome comprising a prophage. In some versions, the prophage repressor gene encodes a repressor of the prophage in the host cell. In some versions, the prophage repressor gene encodes a protein comprising a sequence at least 80% identical, at least 85% identical, at least 90% identical, at least 95% identical, at least 99% identical, or 100% identical to a sequence selected from the group consisting of SEQ ID NOS:2 and 4, and the host cell is L. reuteri.

In some versions, the one or more plasmid maintenance genes comprises a sucrose phosphorylase gene. Sucrose phosphorylase genes are genes that encode enzymes having the activity of EC 2.4.1.7. A large number of sucrose phosphorylase genes are known in the art. See, e.g., Reid et al. 2005 (Reid S J, Abratt V R. Sucrose utilisation in bacteria: genetic organisation and regulation. Appl Microbiol Biotechnol. 2005 May; 67(3):312-21).

In some versions, the sucrose phosphorylase gene encodes a protein comprising a sequence at least 80% identical, at least 85% identical, at least 90% identical, at least 95% identical, at least 99% identical, or 100% identical to SEQ ID NO:6. An exemplary coding sequence of SEQ ID NO:6 is SEQ ID NO:5.

In some versions, the host cell in the system comprises a mutation that reduces sucrose phosphorylase activity with respect to a native form of the host cell. “Sucrose phosphorylase activity” as used herein refers to the activity of EC 2.4.1.7. In some versions, the host cell comprises an inactivating mutation to a native sucrose phosphorylase gene present in a native form of the host cell. In some versions, the native sucrose phosphorylase gene encodes a protein comprising a sequence at least 80% identical, at least 85% identical, at least 90% identical, at least 95% identical, at least 99% identical, or 100% identical to SEQ ID NO:6. In some versions, the host cell is L. reuteri and the host cell encodes a protein of SEQ ID NO:6.

In some versions, the one or more plasmid maintenance genes comprises an alcohol/aldehyde dehydrogenase gene. Alcohol/aldehyde dehydrogenase genes are genes that encode bifunctional enzymes having alcohol dehydrogenase activity (EC 1.1.1.1) and aldehyde dehydrogenase activity (EC 1.2.1.3). A large number of alcohol/aldehyde dehydrogenase genes are known in the art. Examples include the adhE gene of Escherichia coli and homologs thereof. See, e.g., Pony et al. 2020 (Pony P, Rapisarda C, Terradot L, Marza E, Fronzes R. Filamentation of the bacterial bi-functional alcohol/aldehyde dehydrogenase AdhE is essential for substrate channeling and enzymatic regulation. Nat Commun. 2020 Mar. 18; 11(1):1426).

In some versions, the alcohol/aldehyde dehydrogenase gene encodes a protein comprising a sequence at least 80% identical, at least 85% identical, at least 90% identical, at least 95% identical, at least 99% identical, or 100% identical to SEQ ID NO:6. An exemplary coding sequence of SEQ ID NO:8 is SEQ ID NO:7.

In some versions, the host cell in the system comprises a mutation that reduces alcohol/aldehyde dehydrogenase activity with respect to a native form of the host cell. “Alcohol/aldehyde dehydrogenase activity” as used herein refers to the combination of alcohol dehydrogenase activity (EC 1.1.1.1) and aldehyde dehydrogenase activity (EC 1.2.1.3). In some versions, the host cell comprises an inactivating mutation to a native alcohol/aldehyde dehydrogenase gene present in a native form of the host cell. In some versions, the native alcohol/aldehyde dehydrogenase gene encodes a protein comprising a sequence at least 80% identical, at least 85% identical, at least 90% identical, at least 95% identical, at least 99% identical, or 100% identical to SEQ ID NO:8. In some versions, the host cell is L. reuteri and the host cell encodes a protein of SEQ ID NO:8.

The plasmids in the systems of the invention can also include a gene of interest configured to express in the host cell. “Gene of interest” can include any gene that is desired to be included on the plasmid, such as a gene configured to express any desirable gene product. The gene of interest can be configured to constitutively express in the host cell or inducibly express in the host cell.

The gene of interest can be a recombinant gene. The recombinant gene of interest can comprise a coding sequence operably linked to a promoter. The promoter can be heterologous to the coding sequence. In some versions, the promoter is a constitutive promoter. In some versions, the promoter is an inducible promoter.

In some versions, the gene of interest is configured to express a biologic. As used herein, “biologic” refers to any biologically active product capable of being expressed from a gene. The biologic can be biologically active in vivo in any prokaryote or eukaryote or in vitro in any in vitro biochemical system. The biologic can have any activity, whether enzymatic, binding, structural, etc. Biologics that have a therapeutic effect activity are referred to herein as “therapeutic biologics.” Therapeutic biologics can target and promote growth of beneficial cells in the subject, target and inhibit growth of deleterious cells in the subject, target certain cells for destruction, or can have any other activity that provides a therapeutic effect to a subject to which they are introduced.

Examples of biologics include nucleic acids and polypeptides.

Exemplary nucleic acid biologics include DNA and RNA. Preferred nucleic acid biologics include therapeutic nucleic acids. Nucleic acid biologics can generally be classified as nucleotides and nucleosides, oligonucleotides, or polynucleotides. Various types of nucleic acid biologics include oligonucleotides for antisense and antigene applications, DNA aptamers, antisense oligodeoxynucleotides, DNAzymes, DNA vaccines, RNA-based therapeutics, RNA aptamers, RNA Decoys, antisense RNA, ribozymes, small interfering RNAs, and microRNAs, among others.

Suitable polypeptide biologics can include any polypeptide of interest. The polypeptide can have any of a number of amino acid chain lengths. In some versions, the polypeptide can have an amino acid chain length of from about 2 to about 2,000 amino acids, from about 2 to about 1,000 amino acids, from about 2 to about 500 amino acids, from about 3 to about 250 amino acids, or from about 3 to about 225 amino acids. The polypeptide can have a net positive charge at neutral pH, a net negative charge at neutral pH, or a net neutral charge at neutral pH. The polypeptide is preferably soluble in water. The polypeptide can form a globular or fibrous structure or can have an intrinsically disordered structure.

The polypeptide can have any of a number of functionalities. The polypeptide, for example, can be enzymatic or non-enzymatic. The polypeptide can be fluorescent or non-fluorescent. The polypeptide can be a cytokine, a hormone, an antibody, an antimicrobial peptide, and an antigenic peptide, among others.

Exemplary classes of cytokines include interleukins, lymphokines, monokines, interferons (IFNs), colony stimulating factors (CSFs), among others. Specific exemplary cytokines include IL-1 alpha (IL1a), IL-1 beta (IL1b), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-35, IL-36, IFN-alpha, IFN-beta IFN-gamma, TNF-alpha, TNF-beta, CNTF (C-NTF), LIF, OSM (oncostatin-M), EPO (erythropoietin), G-CSF (GCSF), GM-CSF (GMCSF), M-CSF (MCSF), SCF, GH (growth hormone), PRL (prolactin), aFGF (FGF-acidic), bFGF (FGF-basic), INT-2, KGF (FGF7). EGF, TGF-alpha, TGF-beta, PDGF, betacellulin (BTC), SCDGF, amphiregulin, and HB-EG, among others.

Exemplary hormones include epinephrine, melatonin, triiodothyronine, thyroxine, amylin (or islet amyloid polypeptide), adiponectin, adrenocorticotropic hormone (or corticotropin), angiotensinogen, angiotensin, antidiuretic hormone (or vasopressin, arginine vasopressin), atrial-natriuretic peptide (or atriopeptin), brain natriuretic peptide, calcitonin, cholecystokinin, corticotropin-releasing hormone, cortistatin, encephalin, endothelin, erythropoietin, follicle-stimulating hormone, galanin, gastric inhibitory polypeptide, gastrin, ghrelin, glucagon, glucagon-like peptide-1, gonadotropin-releasing hormone, growth hormone-releasing hormone, hepcidin, human chorionic gonadotropin, human placental lactogen, growth hormone, inhibin, insulin, insulin-like growth factor (or somatomedin), leptin, lipotropin, luteinizing hormone, melanocyte stimulating hormone, motilin, orexin, oxytocin, pancreatic polypeptide, parathyroid hormone, pituitary adenylate cyclase-activating peptide, prolactin, prolactin releasing hormone, relaxin, renin, secretin, somatostatin, thrombopoietin, thyroid-stimulating hormone (or thyrotropin), thyrotropin-releasing hormone, and vasoactive intestinal peptide, among others.

Other physiologically active peptides include tachykinin peptides, such as substance P, kassinin, neurokinin A, eledoisin, and neurokinin B; peptide PHI 27 (peptide histidine isoleucine 27); pancreatic polypeptide-related peptides, such as NPY (neuropeptide Y), PYY (peptide YY), and APP (avian pancreatic polypeptide); opioid peptides, such as proopiomelanocortin (POMC) peptides and prodynorphin peptides; AGG01; B-type natriuretic peptide (BNP); lactotripeptides; and peptides that inhibit PCSK9 (Zhang et al. 2014).

Other physiological peptides include the peptides, derivatives, and homologs as described in Wang et al. 2023.

Exemplary antibodies include single-chain antibodies, single-domain antibodies (sdAbs), and single-chain variable fragments (scFvs).

Exemplary antimicrobial peptides include cathelicidins, defensins, protegrins, mastoparan, poneratoxin, cecropin, moricin, melittin, magainin, dermaseptin, nisin, and others. Other antimicrobial peptides include regIII-β and reg-III-γ, which are eukaryotic antimicrobial peptides produced in the intestine. Lactic acid bacteria are well known for their extensive heterogenic repertoire of antimicrobial compounds, including bacteriocins (Alvarez-Sieiro et al. 2016).

Other exemplary biologics include enzymes involved in the production of secondary metabolites having antimicrobial activity, such as polyketides.

Other exemplary biologics include any of a number of antimicrobials. Lactic acid bacteria, for example, are well-known for their extensive heterogenic repertoire of antimicrobial compounds, including bacteriocins (Alvarez-Sieiro et al. 2016). Bacteriocins are small ribosomally synthesized peptides that can inhibit or kill bacteria. The functional diversity of this family of antimicrobials is large, which is illustrated by the fact that bacteriocins can collectively target a wide-array of Gram-negative and Gram-positive bacteria (Cotter et al. 2013). Although narrow-spectrum bacteriocins may be preferential, the application of broad-spectrum bacteriocins may be useful to alleviate bacterial infections of unknown sources. Bacteriocin-mediated impact on the gut microbiota composition can be substantial. This was demonstrated for Abp118, a broad-spectrum bacteriocin produced by L. salivarius UCC118 (Riboulet-Bisson et al. 2012). See also Corr et al. 2007 (Corr S C, Li Y, Riedel C U, O'Toole P W, Hill C, Gahan C G. Bacteriocin production as a mechanism for the antiinfective activity of Lactobacillus salivarius UCC118. Proc Natl Acad Sci USA. 2007 May 1; 104(18):7617-21). By comparing the microbiota in mice and pigs between groups that 5 were administrated with L. salivarius wild-type or L. saiivariusΔabp118, it was confirmed that the presence of the bacteriocin-producing lactobacilli alters the gut microbiota composition without significance changes in microbial diversity. See also Kommineni et al. 2015. One example of a useful bacteriocin is nisin, which is produced by select Lactococcus lactis strains and streptococci. The 372 basepair gene encoding nisin (nisA) is one of the six natural nisin variants, and certain mutants NisA display enhanced activity against Gram-positive and Gram-negative pathogens (Field et al. 2008, Field et al. 2012).

Other exemplary biologics comprise lytic biologics. As used herein, “lytic biologic” refers to any biologic that causes or aids, either directly or indirectly, the lysis of a cell in which it is produced. Expression of a lytic biologic in a cell, for example, can induce lysis of the cell and any contents thereof, including any other biologics made by the cell.

Lytic biologics comprise lytic proteins. Lytic proteins are well known in the art. A number of lytic proteins, for example, are found in bacteriophages and serve to lyse cells during the lytic stages of the bacteriophage's life cycle. These include holins and lysins (Sheehan et al. 1999). During bacteriophage replication, biologically active lysins are present in the cytosol but require expression of a membrane protein, holin, to release the virions from the cell. When holin levels are optimal, the lysin can access the peptidoglycan layer for cleavage which leads to bacterial cell lysis (Wang et al. 2000). So far, five main groups of lysins have been identified that can be distinguished from one and another based on the cleavage specificity of the different bonds within the peptidoglycan (Fischetti 2009). Structurally, lysins can comprise a single catalytic domain, which generally is typical for lysins derived from bacteriophages targeting Gram-negative bacteria (Cheng et al. 1994). Bacteriophages targeting Gram-positive bacteria typically encode lysins that contain multiple domains: a N-terminal catalytic domain and a C-terminal cell-wall binding domain (Nelson et al. 2006, Navarre et al. 1999). A few lysins have been identified that have three domains (Becker et al. 2009).

A number of other lytic proteins are native to the cells themselves (Feliza et al. 2012, Jacobs et al. 1994, Jacobs et al. 1995, López et al. 1997). These lytic proteins can affect cell wall metabolism or introduce nicks in the cell wall. Five protein classes are differentiated by the wall component they attack (Loessner et al. 2005, Loessner et al. 2002).

In some versions, the biologic is a therapeutic biologic and a promoter operably linked to the biologic coding sequence is a promoter inducible by an environmental condition of a disease that the therapeutic biologic is capable of treating.

An inducible promoter operably linked to a coding sequence of the biologic can be an inducible promoter sensitive to an environmental cue or condition, such as sugar concentration, bile acid concentration, or any other condition of the site in which expression of the coding sequence is desired.

In some versions, the biologic comprises a chimeric protein. A chimeric protein is a recombinant protein comprising sequences from two different native polypeptides. Any of the protein biologics described herein (or fragments thereof) can be fused with another polypeptide to generate a chimeric protein biologic.

In some versions, the biologic comprises a protein comprising an affinity tag. The affinity tags can be used for purification, detection with antibodies, or other uses. A number of affinity tags are known in the art. Exemplary affinity tags include the His tag, the Strep II tag, the T7 tag, the FLAG tag, the S tag, the HA tag, the c-Myc tag, the dihydrofolate reductase (DHFR) tag, the chitin binding domain tag, the calmodulin binding domain tag, the cellulose binding domain tag, and the HiBiT tag. The sequences of each of these tags are well-known in the art.

In some versions, the biologic is a fusion protein comprising a label. A label is a polypeptide sequence that is capable of being detected by any of a number methods. The label can be a fluorescent label (e.g., GFP, RFP, etc.), an enzymatic label (horseradish peroxidase (HRP), alkaline phosphatase (AP), glucose oxidase and β-galactosidase), an antibody, an antigen, or other types of polypeptide labels that can be fused to another polypeptide for detection.

In some versions, the plasmid is devoid of any antibiotic resistance gene. Antibiotic resistance genes are well known in the art and are commonly used on plasmids for plasmid maintenance. See, e.g., Jian et al. 2021 (Jian Z, Zeng L, Xu T, Sun S, Yan S, Yang L, Huang Y, Jia J, Dou T. Antibiotic resistance genes in bacteria: Occurrence, spread, and control. J Basic Microbiol. 2021 December; 61(12):1049-1070). Resistance genes can be divided into the following categories based on the class of antibiotics they grant resistance to which include tetracyclines (tet), sulfonamides (sul), P-lactams (bla), macrolides (erm), aminoglycosides (aac), fluoroquinolone (fca), colistin (mcr), vancomycin (van), and multidrug (mdr). Exemplary antibiotic resistance genes include bla_{TEM}, mecA, bla_{CTX-M-1,9}, bla_{CTX-M-25}, bla_{TEM-116,229}, bla_{CTX-M9}, bla_{CTX-M-15,33,109,114}, bla_{TEM-116}, bla_{CTX-M}, bla_{PSE}, bla_{CMY-2}, bla_{HRV-1}, bla_{HRVM-1}, bla_{KPC}, bla_{OXA-48-like}, bla_{NDM}, bla_{TEM}, bla_{CTX-M-1,9}, bla_{OXA-48}, bla_{VIM}, mecA, bla_{CTX-M}, qnrA, qnrS, aac(6′)-Iu,Is,Iy, qnrA3, aac(6′)-Ib-cr, aac(6′)-Ib-cr, VanY, dfrB2, sul1, sul2, tetW, tetM, ermB, and ermF, among others.

Another aspect of the invention is directed to methods of using the systems of the invention. In some versions, the methods comprise introducing a plasmid of the invention into a host cell of the invention. In some versions, the methods comprise growing a host cell of the invention with a plasmid of the invention comprised within the host cell. In some versions, the methods comprise expressing the gene of interest from the plasmid within the host cell.

In some versions, the plasmid in the system comprises a sucrose phosphorylase gene, the host cell in the system comprises a mutation that reduces sucrose phosphorylase activity with respect to a native form of the host cell and/or comprises an inactivating mutation to a native sucrose phosphorylase gene present in the native form of the host cell, and the growing comprises growing the host cell in the presence of sucrose. In some versions, the growing comprises growing the host cell growing the host cell in the presence of sucrose as a sole carbon source.

In some versions, the plasmid in the system comprises an alcohol/aldehyde dehydrogenase gene, the host cell in the system comprises a mutation that reduces alcohol/aldehyde dehydrogenase activity with respect to a native form of the host cell and/or comprises an inactivating mutation to a native alcohol/aldehyde dehydrogenase gene present in the native form of the host cell, and the growing comprises growing the host cell in the presence of glucose. In some versions, the growing comprises growing the host cell in the presence of glucose as a sole carbon source.

Some methods of the invention comprise growing the host cell comprising the plasmid therein in vivo (e.g., within another organism). Such methods can comprise growing the host cell in vivo in a gastrointestinal tract of a subject. Some methods of the invention comprise administering the host cell comprising the plasmid therein to a subject and growing the host cell in vivo. Such methods can comprise orally administering the host cell to a subject.

In some versions, the plasmid in the system comprises a sucrose phosphorylase gene, and/or the host cell in the system comprises a mutation that reduces sucrose phosphorylase activity with respect to a native form of the host cell, and/or the host cell in the system comprises an inactivating mutation to a native sucrose phosphorylase gene present in the native form of the host cell, and/or the methods comprise orally administering the host cell comprising the plasmid therein to a subject, and/or the methods comprise growing the host cell comprising the plasmid therein in vivo (such as in the gastrointestinal tract of the subject), and/or the methods comprise orally administering sucrose to the subject.

In some versions, the plasmid in the system comprises an alcohol/aldehyde dehydrogenase gene, and/or the host cell in the system comprises a mutation that reduces alcohol/aldehyde dehydrogenase activity with respect to a native form of the host cell, and/or the host cell in the system comprises an inactivating mutation to a native alcohol/aldehyde dehydrogenase gene present in the native form of the host cell, and/or the methods comprise orally administering the host cell comprising the plasmid therein to a subject, and/or the methods comprise growing the host cell comprising the plasmid therein in vivo (such as in the gastrointestinal tract of the subject), and/or the methods comprise orally administering glucose to the subject.

Some methods of the invention comprise growing the host cell comprising the plasmid therein in vitro.

The host cells of the invention with the plasmids of the invention contained therein are referred to herein as “transformed host cells” of the invention.

The transformed host cells of the invention can be administered to a subject. The subject can include any animal, such as mammals or humans. The transformed host cells can be administered orally, nasally, rectally, or via any other means of administration.

The expression systems of the invention can be used to introduce a biologic to a site. The site to which the biologic is introduced can be any site in which it is desired to introduce or produce the biologic. In some versions, the site is an in vitro site, for example, for producing the biologic for subsequent use (either in vitro or in vivo). Exemplary in vitro sites include test tubes, petri dishes, high-throughput device wells, bioreactors, etc.

In some versions, the site is an in vivo site. The in vivo site can be any site on or in a subject's body. The subject can be an animal, such as a mammal or a human.

In some versions, the site comprises a gastrointestinal tract of a subject. The methods of introducing the biologic to the site in such versions can comprise administering the transformed host cell to the gastrointestinal tract. The transformed host cell can be administered to the gastrointestinal tract by any method known in the art. The transformed host cell can be administered orally, rectally, or directly into the gastrointestinal tract via a stoma. The transformed host cell is preferably administered directly into or upstream of the small intestines, so that the transformed host cell ultimately passes through or into the small intestines. The transformed host cell can be swallowed or introduced via a tube. The transformed host cell can be combined in a composition with a pharmaceutically acceptable excipient, carrier, buffer, stabilizer or other material well known to those skilled in the art. Such materials should be non-toxic and should not interfere with the efficacy of the transformed host cell. The precise nature of the carrier or other material may depend on the route of administration. The composition can be liquid, solid, or semi-solid. The composition can comprise a foodstuff or can take the form of a pharmaceutical composition. Those of relevant skill in the art are well able to prepare suitable compositions. In some versions, biologics introduced to the gastrointestinal tract can be delivered systemically within the organism. See, e.g., Wang et al. 2023 and U.S. Pat. No. 10,376,563.

Expression of the biologic can be induced during, after, or prior to administering the transformed host cell to the site. Inducing expression of the biologic during administration to the site can be accomplished, for example, by co-administering an inducer with the transformed host cell in a single composition or simultaneously administering (whether in separate compositions or in a single composition) the inducer and the transformed host cell. In this manner, expression of the biologic can be initiated during the administration for subsequent introduction to the site.

Inducing expression of the biologic after administration to the site can be accomplished by administering an inducer after the transformed host cell is administered and, preferably, reaches the site. Depending on the type of transformed host cell and site, the transformed host cell can survive and/or proliferate at the site for a period until the inducer is administered. Administration of the inducer then induces expression of the biologic and introduction of the biologic to the site.

In some versions of the invention, one or more of the biologics can be introduced to a site without inducing lysis of the transformed host cell. In the case of polypeptide biologics, for example, the transformed host cell can comprise a recombinant gene configured to express and secrete the polypeptide. Elements for engineering a transformed host cell to secrete a polypeptide are well known in the art. Typical elements include a signal peptide-encoding sequence placed upstream of—and in-frame with—the coding sequence of the polypeptide to be secreted. The sequences of a large number of signal peptides for bacteria are known in the art. Exemplary signal peptide sequences are available on the world wide web at cbs.dtu.dk/services/SignalP/. The signal peptide can be cleaved from or remain intact on the polypeptide after secretion. The secreted polypeptide can be expressed from a coding sequence comprised within the regulatory sequence.

“Reduce” used with respect to reducing an activity in a cell or reducing the activity of a gene refers to any diminishment of activity to any degree, including complete ablation of the activity.

“Inactivating mutation” refers to any mutation that reduces the activity of a gene. “Activity of a gene” as used herein refers to transcription of the gene, translation of an mRNA from the gene, production of a protein from the gene, or activity of a protein expressed from the gene. Exemplary inactivating mutations include point mutations, substitutions, deletions (complete or partial), and insertions. The inactivating mutation can be anywhere in the genome of the cell. In some versions, the inactivating mutation of a particular gene is in the gene itself or is a deletion of the entire gene itself.

A “native gene” in a “native form of a host cell” refers to a form of a gene that does not include a mutation, such as an inactivating mutation, as described herein. A host cell that includes an inactivating mutation to a native gene present in a native form of the host cell refers to any mutation to what would otherwise be the native gene in the native host cell.

The transformed host cell of the invention can be engineered using any methods known in the art. General methods are provided in Green et al. 2012 (Green et al., Molecular Cloning: A Laboratory Manual, 4th ed., Cold Spring Harbor Laboratory Press, 2012). Methods for engineering lactic acid bacteria such as L. lactis are provided by van Pijkeren and Britton et al. 2012 (van Pijkeren J P, Britton R A. High efficiency recombineering in lactic acid bacteria. Nucleic Acids Res. 2012 May; 40(10):e76), van Pijkeren and Neoh et al. 2012 (van Pijkeren J-P, Neoh K M, Sirias D, Findley A S, Britton R A. 2012. Exploring optimization parameters to increase ssDNA recombineering in Lactococcus lactis and Lactobacillus reuteri. Bioengineered 3:209-217), Oh et al. 2014 (Oh J H, van Pijkeren J P. CRISPR-Cas9-assisted recombineering in Lactobacillus reuteri. Nucleic Acids Res. 2014; 42(17):e131), Barrangou et al. 2016 (Barrangou R, van Pijkeren J P. Exploiting CRISPR-Cas immune systems for genome editing in bacteria. Curr Opin Biotechnol. 2016 February; 37:61-8), and Zhang et al. 2018 (Zhang S, Oh J H, Alexander L M, Özgam M, van Pijkeren J P. d-Alanyl-d-Alanine Ligase as a Broad-Host-Range Counterselection Marker in Vancomycin-Resistant Lactic Acid Bacteria. J Bacteriol. 2018 Jun. 11; 200(13):e00607-17).

“Corresponding microorganism” refers to a microorganism of the same species having the same or substantially the same genetic and proteomic composition as a transformed host cell of the invention, with the exception of genetic and proteomic differences resulting from the modifications specified for the transformed host cells of the invention. In some versions, the corresponding microorganism is the native version of the transformed host cell of the invention, i.e., the unmodified microorganism as found in nature. The terms “microorganism” and “microbe” are used interchangeably herein. “Corresponding native microorganism” refers to a native microorganism from which the transformed host cell is either directly or indirectly derived. The corresponding native microorganism will typically be a native microorganism having the closest genetic structure (e.g., highest percent genomic sequence identity) to the transformed host cell.

“Heterologous” as used herein refers to an element in an arrangement with another element that does not occur in nature. For example, a gene or protein that is heterologous to a given cell is a gene or protein that does not occur in the cell in nature. A promoter that is heterologous to a given coding sequence is a promoter that is not operably linked to the coding sequence in nature. A secretion signal sequence that is heterologous to a given protein (such as an enzyme) is a secretion signal sequence that is not operably linked with the protein in nature.

“Recombinant” as used herein with respect to a cell, such as a host cell of the invention, refers to a microorganism that comprises a recombinant nucleic acid, a recombinant gene, or a recombinant polypeptide. “Recombinant” as used herein with respect to a recombinant nucleic acid or polypeptide refers to a nucleic acid or polypeptide comprising a sequence that is not naturally occurring. “Recombinant” as used herein with respect to a recombinant gene refers to a gene that comprises a recombinant nucleic acid sequence, is present within a cell in which it does not naturally occur, and/or is present at a locus (e.g., genetic locus or on an extrachromosomal plasmid) that is different than the locus in which it is present in a corresponding native cell.

“Growing” as used herein with respect to growing a host cell is used synonymously as “culturing,” and refers to subject a host cell to conditions suitable for cell division.

Growing the host cell in the presence of one or more substrates (e.g., sugars such as sucrose and/or glucose) as a “sole carbon source” means growing the host cell under conditions such that removal of the one or more substrates defined as a sole carbon source would generate a condition unsuitable for cell growth. Thus, growing the host cell in the presence of one or more substrates (e.g., sugars such as sucrose and/or glucose) as a sole carbon source does not necessarily exclude the presence of other carbon sources, but it does exclude the presence of other carbon sources in amounts that would be sufficient to support growth in the absence of the substrate(s) named as the sole carbon source(s). Examples of various carbon sources that can be excluded or included in amounts that do not support host cell growth include adonitol, arabinose, arabitol, ascorbic acid, chitin, D-cellubiose, 2-deoxy-D-ribose, dulcitol, (S)-(+)-erythrulose, fructose, fucose, galactose, glucose (in some embodiments when sucrose is a sole carbon source), inositol, lactose, lactulose, lyxose, maltitol, maltose, maltotriose, mannitol, mannose, melezitose, melibiose, microcrystalline cellulose, palatinose, pentaerythritol, raffinose, rhamnose, ribose, sorbitol, sorbose, starch, sucrose (in some embodiments when glucose is a sole carbon source), trehalose, xylitol, xylose, and hydrates thereof.

“Gene” minimally refers to a coding sequence configured for expression in a host cell. The coding sequence, for example can be operably linked to a promoter. A gene can optionally include other genetic elements that facilitate or regulate transcription and/or translation of the coding sequence. Such genetic elements can include enhancers and ribosome binding sites (RBSs), among other elements.

“Coding sequence” as used herein refers to a nucleic acid sequence in a gene that encodes a gene product. The term “coding sequence” encompasses sequences that include codons that are ultimately transcribed and translated into polypeptides as well as sequences that do not include codons and/or are merely transcribed (e.g., antisense RNA, etc.).

“Gene product” as used herein refers to any product resulting from expression (e.g., transcription or transcription and translation) of a gene. The term “gene product” explicitly encompasses polypeptides as well as nucleic acids such as RNA (e.g., mRNA, pri-microRNA, pre-microRNA, microRNA, antisense RNA (asRNA) etc.) and DNA (cDNA).

“Promoter” is used herein as understood in the art and typically refers to a nucleic acid sequence that confers transcription of an operably linked coding sequence. The promoters of the invention can comprise any promoter capable of being employed in the transformed host cells of the invention. Promoters suitable for use in bacteria are typically derived from microbial or viral sources. Exemplary promoters include but are not limited to: promoters capable of recognizing the T4, T3, Sp6, and T7 polymerases; the P_{R} and P_{L} promoters of bacteriophage lambda; the trp, recA, heat shock, and lacZ promoters of E. coli; the alpha-amylase and the sigma-specific promoters of B. subtilis; the promoters of the bacteriophages of Bacillus; Streptomyces promoters; the int promoter of bacteriophage lambda; the bla promoter of the beta-lactamase gene of pBR322; and the CAT promoter of the chloramphenicol acetyl transferase gene. Prokaryotic promoters are reviewed by Glick, J. Ind. Microbiol. 1:277 (1987); Watson et al, Molecular Biology of the Gene, 4th Ed., Benjamin Cummins (1987); and Sambrook et al., In: Molecular Cloning: A Laboratory Manual, 3^{rd} ed., Cold Spring Harbor Laboratory Press (2001).

Any promoter of the invention can be an inducible promoter. “Inducible promoter” as used herein refers to a regulated promoter that is active only in response to specific stimuli. Such specific stimuli are referred to herein as “inducers.” Exemplary inducers include proteins, metabolites, chemicals, and culture conditions. In some versions, the inducer is a particular concentration of a particular protein, metabolite, chemical, or culture condition. In some versions, the inducer is the presence of a particular protein, metabolite, chemical, or culture condition. In some versions, the inducer is the absence of a particular protein, metabolite, chemical, or culture condition. Exemplary inducible promoters include but are not limited to the lac promoter (regulated by IPTG or analogs thereof), the lacUV5 promoter (regulated by IPTG or analogs thereof), the tac promoter (regulated by IPTG or analogs thereof), the trc promoter (regulated by IPTG or analogs thereof), the araBAD promoter (regulated by L-arabinose), the phoA promoter (regulated by phosphate starvation), the recA promoter (regulated by nalidixic acid), the proU promoter (regulated by osmolarity changes), the cst-1 promoter (regulated by glucose starvation), the tetA promoter (regulated by tetracycline), the cadA promoter (regulated by pH), the nar promoter (regulated by anaerobic conditions), the P_{L} promoter (regulated by thermal shift), the cspA promoter (regulated by thermal shift), the T7 promoter (regulated by thermal shift), the T7-lac promoter (regulated by IPTG), the T3-lac promoter (regulated by IPTG), the T5-lac promoter (regulated by IPTG), the T4 gene 32 promoter (regulated by T4 infection), the nprM-lac promoter (regulated by IPTG), the VHb promoter (regulated by oxygen), the metallothionein promoter (regulated by heavy metals), the MMTV promoter (regulated by steroids such as dexamethasone) and variants thereof.

Any promoter of the invention can be a constitutive promoter. “Constitutive promoter” as used herein refers to a promoter that is constitutively active, i.e., is not regulated by an inducer. Suitable constitutive promoters are known in the art and include constitutive adenovirus major late promoter, a constitutive MPSV promoter, and a constitutive CMV promoter.

“Operably linked” as used herein generally refers to a connection of two genetic elements in a manner wherein one can operate on or have effects on the other. “Operably linked” used in reference to a promoter and a coding sequence refers to a connection between the promoter and the coding sequence such that the coding sequence is under transcriptional control of the promoter. For example, promoters are generally positioned 5′ (upstream) of a coding sequence to be operably linked to the promoter. In the construction of heterologous promoter/coding sequence combinations, it is generally preferred to position the promoter at a distance from the transcription start site that is approximately the same as the distance between that promoter and the coding sequence it controls in its natural setting, i.e., in the gene from which the promoter is derived. As is known in the art, some variation in this distance can be accommodated without loss of promoter function.

A gene “configured to express” a particular gene product in a host cell refers to the gene having a coding sequence encoding the gene product any other genetic elements sufficient for transcription (for nucleic acid gene products) or transcription and translation (for protein gene products) of the coding sequence. Exemplary genetic elements include promoters, enhancers, ribosome binding sites (RBSs), etc.

“Overexpress” refers to the increased production of a gene product from a gene compared to an endogenous or basal product rate for that gene product. Methods of testing for overexpression are well known in the art, for example transcribed RNA levels can be assessed using RT-PCR and protein levels can be assessed using SDS-PAGE gel analysis.

“Introduce” as used herein with respect to introducing an element such as a microorganism or a biologic to a site, such as an in vitro site or an in vivo site (such as a gastrointestinal tract), refers to any activity that results in the initial appearance or increased appearance of the element at a given site. Introducing a microorganism to a site can comprise, for example, inoculating, administering, culturing, and growing the microorganism at that site. Introducing a biologic to a site can comprise, for example, stimulating production of the biologic in the microorganism and/or releasing the biologic (e.g., through cell lysis or secretion) at the site. “Introduce” as used herein with respect to introducing an element such as a plasmid of the invention within a host cell of the invention refers to any activity that results in the initial appearance or increased appearance of the element in the host cell, such as transforming a host cell with a plasmid.

“Devoid of any antibiotic resistance gene” refers to lacking a functional gene capable of conferring antibiotic resistance.

The terms “identical,” “identity,” etc. in the context of two or more nucleic acid or polypeptide sequences, refer to two or more sequences or subsequences that are the same or have a specified percentage of amino acid residues or nucleotides that are the same, when compared and aligned for maximum correspondence, as measured using a sequence comparison algorithm available to a person of skill in the art.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see Current Protocols in Molecular Biology, F. M. Ausubel et al., eds., Current Protocols, a joint venture between Greene Publishing Associates, Inc. and John Wiley & Sons, Inc., (supplemented through 2008)).

One example of an algorithm that is suitable for determining percent sequence identity and sequence similarity for purposes of defining homologs is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001. The above-described techniques are useful in identifying homologous sequences for use in the methods described herein.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### Examples

### Introduction

To address the concern of antibiotic resistance genes in plasmids, we have developed three antibiotic-free plasmid selection systems. Our model strain for these systems is Limosilactobacillus reuteri VPL1014 (L. reuteri) (Alexander et al. 2019), which contains two phage repressors (Tables 1 and 2) in two prophage genomes.

### Results and Discussion

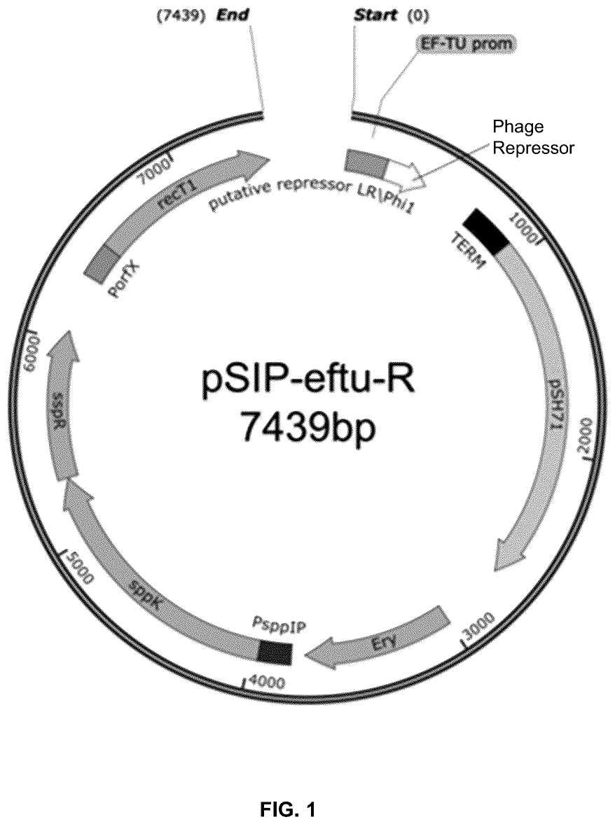

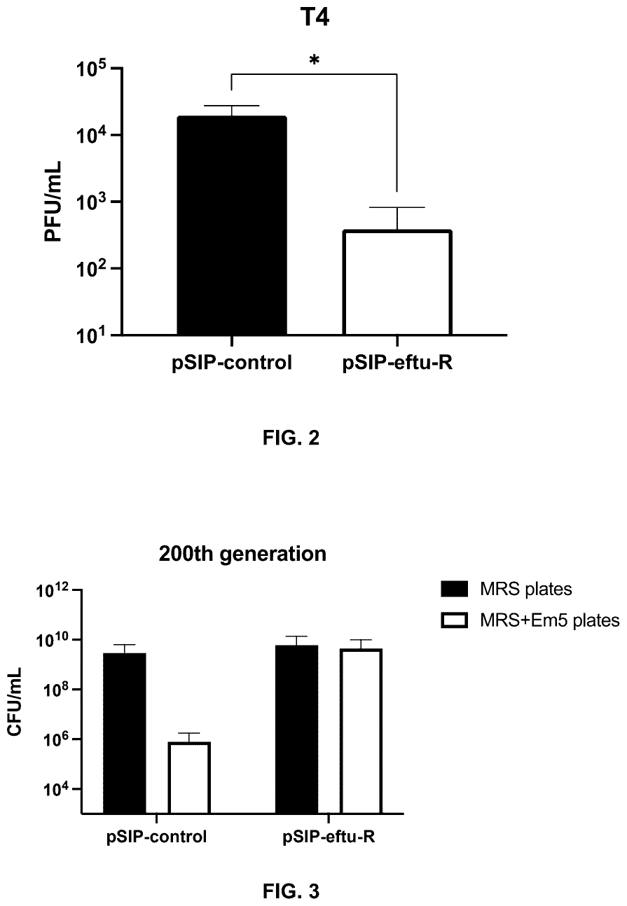

The first system we developed involves L. reuteri which harbors a derivative of plasmid pSIP411 encoding a phage repressor 1 (repressor) protein (pSIP-eftu-R; FIG. 1). The repressor protein represses induction of prophages from the L. reuteri chromosome (FIG. 2). We discovered that plasmid-expression of the phage repressor provides a strong selective pressure, which resulted plasmid maintenance for at least 200 generations in antibiotic-free media (FIG. 3).

We will test repressor-dependent plasmid stability in a second microbe, the lactic acid bacterium (LAB) Lactiplantibacillus plantarum WCFS1 strain. The WCFS1 strain and its bacteriophages are well-studied relative to the other LAB type-strains. The chromosome of WCFS1 contains four prophages (two intact and two incomplete phages) that are similar in phage composition to L. reuteri 6475. Repressors in these prophages were previously identified (Ventura et al. 2003). Therefore, we can clone one or more WCFS1 repressors in the same pSIP411 vector and transform WCFS1 to test plasmid stability. We predict that the WCFS1 repressors will result in enhanced plasmid retention.

We will also test repressor-dependent plasmid stability with prophage repressors in host cells that do not have a cognate prophage. In these experiments, the L. reuteri repressor 1 and/or the L. reuteri repressor 2 will be included in a plasmid that is used to transform a host cell other than L. reuteri, such as Lactiplantibacillus plantarum WCFS1 or a host cell that does not have a prophage, and plasmid stability will be tested. Lactiplantibacillus plantarum WCFS1 repressors will be included in a plasmid that is used to transform a host cell other than Lactiplantibacillus plantarum WCFS1, such as L. reuteri or a host cell that does not have a prophage, and plasmid stability will be tested. We predict that the repressors will result in enhanced plasmid retention in host cells that do not have a cognate prophage.

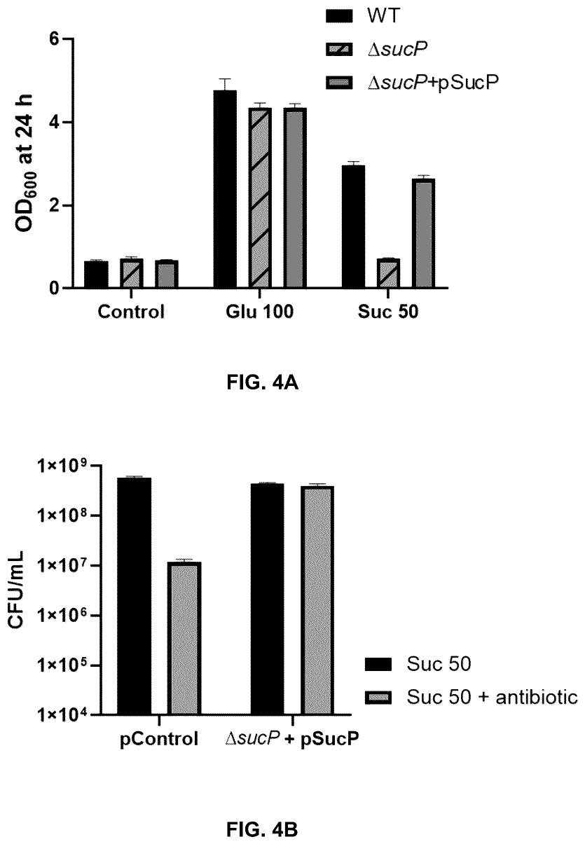

In the second system, we constructed L. reuteriΔsucP. The gene sucP encodes the enzyme sucrose phosphorylase, which hydrolyzes sucrose. Deletion of sucP means L. reuteri cannot grow in media with sucrose as a sole carbon source (Suc 50) (FIG. 4A). By cloning the sucP gene into a plasmid, ΔsucP strain retained the plasmid in Suc 50 (FIG. 4B).

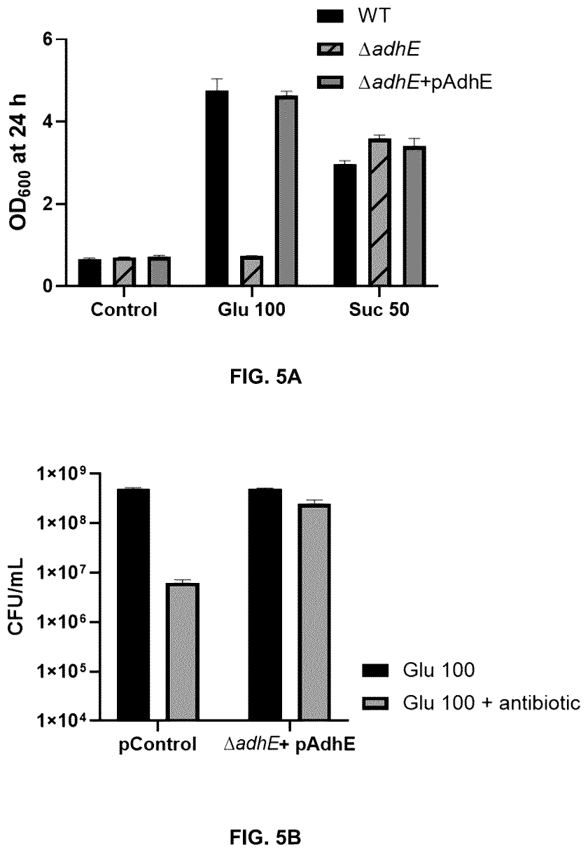

Analogously, in the third system, when we inactivated the gene encoding the bifunctional alcohol/aldehyde dehydrogenase (AdhE) enzyme in L. reuteri (L. reuteri ΔadhE), the mutant cannot grow in media with glucose as a sole carbon source (Glu 100) (FIG. 5A). However, by inserting the adhE gene into the plasmid, we restored the function of adhE and ensure stable maintenance of the plasmid in Glu 100 (FIG. 5B). These antibiotic-free plasmid selection systems provide alternative methods for selecting and maintaining plasmids without relying on antibiotic resistance genes.

The application of our antibiotic-free plasmids holds significant advantages in various fields of research and biotechnology.

The cloning of sucP or adhE in L. reuteriΔsucP and L. reuteriΔadhE, respectively, will yield stable maintenance of plasmids without the need for the presence of an antibiotic selection marker. A major advantage is that mouse diets containing sucrose, for example, will result in stable plasmid maintenance through the gastrointestinal transit without significant disturbance of the gut microbiome. Unlike antibiotics, our plasmid maintenance system allows biological studies in the context of a complex and complete microbiome. In general, antibiotic-free plasmids are extensively used in biomedical research for heterologous protein expression, including therapeutics. Plasmid-based expression has the added advantage of multi-copy expression of the recombinant protein, therefore increasing yield.

The repressor system not only keeps the repressor-gene the plasmid maintained in the cell, at the same time, prophages are suppressed and thus not induced. This is a major advantage for applications in which (recombinant) cultures are grown in large vessels where prophage induction can be a major liability for quality output. Not only will the repressor system increase microbial survival, it is also expected the plasmid-encoded expression of the repressor will yield more recombinant protein, and it will be a major cost saving approach as removal of antibiotic-containing media belongs to the past.

Overall, our design of the antibiotic-free plasmid systems by regulating the expression of phage repressor or mediating sugar metabolism offers advantages in terms of biosafety, experimental reliability, downstream applications, and strain stability. In addition, any bacterial strain carrying prophages has the potential to utilize the repressor of their phage to develop a strain-specific antibiotic-free plasmid. Furthermore, it is worth noting that the approach of utilizing sucP and adhE genes can be applied more broadly in bacterial strains, regardless of whether they contain phages or not. These benefits make our systems valuable tools in various fields of research and industrial applications.

The antibiotic-free plasmid systems can be employed for the production of recombinant proteins, enzymes or chemical compounds. The presence of repressor on the plasmid can increase the culture stability and avoid the use of antibiotic. In addition, these systems can be applied in the field of probiotics. By incorporating a therapeutic protein on an antibiotic-free plasmid and transferring it into a probiotic organism like L. reuteri, we can create a more stable and controlled probiotic product.

### Methods

Construction of Plasmid pSIP-Eftu-R

The ligation cycling reaction (LCR) method was used to clone the promoter eftu and repressor sequences (eftu-R) into the pSIP411 vector (Mairhofer et al. 2008), resulting in the creation of pSIP-eftu-R. The gene fragment eftu-R was synthesized by GeneWiz (Table 5). PCR amplicons of the plasmid backbone were generated using Phusion Hot Start Polymerase II (Thermo Scientific) with the specified primers (Table 5). Following this, the plasmids were successfully transformed into Lactococcus lactis MG1363, followed by a recovery period in GM17 media at 30° C. for 3 hours, and subsequent plating on GM17 agar supplemented with g/mL erythromycin. After 24-h incubation at 30° C., colony PCR was performed on the erythromycin-resistant colonies to screen for positive transformants, followed by confirmation through Sanger sequencing conducted by GeneWiz. Plasmids exhibiting the correct sequencing were isolated from Lc. lactis cells and transferred to L. reuteri. The cells were anaerobically recovered in MRS for 3 hours, then plated on MRS agar containing 5 g/mL erythromycin, with transformants subsequently confirmed through PCR and sequencing analysis. As a control, the original plasmid pSIP411 was also transformed into L. reuteri wildtype to generate L. reuteri (pSIP-control).

### Phage Production

The L. reuteri ΔΦΔattB1ΔΦ2ΔattB2 strain forms plaques upon exposure to the phage. This strain served as a sensitive lytic host (LH) for determining the plaque-forming units (PFU). The plaque assay procedure followed a previously outlined method (de Kok et al. 2014). In summary, overnight cultures of L. reuteri (pSIP-control) and L. reuteri (pSIP-eftu-R) were inoculated into fresh 40 mL of MRS medium supplemented with 5 g/mL erythromycin to obtain an initial OD of 0.1. Following a 4-hour incubation in a 37° C. water bath, supernatants were harvested and filter sterilized (0.22 m, Millipore). These supernatants were serially diluted using a phage diluent composed of 8.0 mM MgSO4 and 10.0 mM Tris-HCl at pH 7.5. An overnight culture of LH was washed once with an equivalent volume of phage diluent and adjusted to an OD₆₀₀ of 0.5 in the same diluent. Subsequently, the diluted supernatants (200 μL) were combined with 200 μL of the LH cell suspension and 4 μL of 1 M CaCl₂ solution, followed by 1-hour incubation at 37° C. Then, 3 mL of a soft top-agarose solution (0.2%, w/v) containing 10 mM CaCl₂ (preheated to 50° C.) was gently added to the mixture before being poured onto an MRS-agar plate supplemented with 10 mM CaCl₂. The plates were then left to incubate overnight at 37° C., after which the plaques were enumerated.

### Repressor-Dependent Plasmid Stability

The cells of L. reuteri (pSIP-control) and L. reuteri (pSIP-eftu-R) were subcultured in fresh MRS to an OD₆₀₀ of 0.01 every 24 h for a total of 20 passages, maintaining a constant 37° C. incubation temperature throughout. Following the 20th passage, the cultures were subjected to serial dilution and plated on two types of agar plates: MRS agar plates (to determine total cell counts) and MRS agar plates supplemented with erythromycin (to quantify plasmid-carrying cell populations). Plates were anaerobically incubated in a 37° C. hypoxic chamber for 24 hours.

### SucP- and AdhE-Dependent Plasmid Stability

The sucP (Table 3) and adhE (Table 4) from L. reuteri were inserted into pSIP411 vector to yield pSucP and pAdhE, respectively. Subsequently, L. reuteri ΔsucP and ΔadhE mutants were transformed with these plasmids to yield ΔsucP harboring pSucP (ΔsucP+pSucP) and ΔadhE harboring pAdhE (ΔadhE+pAdhE). The ΔsucP+pSucP strain was grown in mMRS supplemented with 50 mM sucrose (Sue 50). The ΔadhE+pAdhE strain was grown in mMRS supplemented with 100 mM glucose (Glu 100). After ten passages in Suc 50 or Glu100 without antibiotics (initial OD₆₀₀ of 0.1, 24 h incubation at 37° C. per passage), serial-diluted cultures were plated on Suc 50 or G100 agar plates supplemented with or without antibiotics and incubated at 37° C. for 24 h in hypoxic condition to determine CFU and plasmid stability in each strain. L. reuteri harboring pSIP411 plasmid with no gene expression cassette (pControl) was used as a control to compare plasmid stability with ΔsucP+pSucP or ΔadhE+pAdhE.

### REFERENCES

Alexander L M, Oh J H, Stapleton D S, Schueler K L, Keller M P, Attie A D, van Pijkeren J P. Exploiting Prophage-Mediated Lysis for Biotherapeutic Release by Lactobacillus reuteri. Appl Environ Microbiol. 2019 May 2; 85(10):e02335-18.
Bennett, Pauline M. “Plasmid encoded antibiotic resistance: acquisition and transfer of antibiotic resistance genes in bacteria.” British journal of pharmacology 153.S1 (2008): S347-S357.
de Kok S, Stanton L H, Slaby T, Durot M, Holmes V F, Patel K G, Platt D, Shapland E B, Serber Z, Dean J, et al. 2014, Rapid and Reliable DNA Assembly via Ligase Cycling Reaction. ACS Synth. Biol. 3 (2), 97-106.
Hall, Barry G. “Predicting the evolution of antibiotic resistance genes.” Nature Reviews Microbiology 2.5 (2004): 430-435.
Huddleston, Jennifer R. “Horizontal gene transfer in the human gastrointestinal tract: potential spread of antibiotic resistance genes.” Infection and drug resistance (2014): 167-176.
Kroll, Jens, et al. “Plasmid addiction systems: perspectives and applications in biotechnology.” Microbial biotechnology 3.6 (2010): 634-657.
Mairhofer, Juergen, and Reingard Grabherr. “Rational vector design for efficient non-viral gene delivery: challenges facing the use of plasmid DNA.” Molecular biotechnology 39 (2008): 97-104.
Oh J H, Alexander L M, Pan M, Schueler K L, Keller M P, Attie A D, Walter J, van Pijkeren J P. 2019. Dietary fructose and microbiota-derived short-chain fatty acids promote bacteriophage production in the gut symbiont Lactobacillus reuteri. Cell Host Microbe 25:273-284.
Ventura M, Canchaya C, Kleerebezem M, de Vos W M, Siezen R J, Brussow H. The prophage sequences of Lactobacillus plantarum strain WCFS1. Virology. 2003 Nov. 25; 316(2):245-55.
Wang Y, Zhu D, Ortiz-Velez L C, Perry J L, Pennington M W, Hyser J M, Britton R A, Beeton C. A bioengineered probiotic for the oral delivery of a peptide Kv1.3 channel blocker to treat rheumatoid arthritis. Proc Natl Acad Sci USA. 2023 Jan. 10; 120(2):e2211977120.

### Sequences

TABLE 1
Coding and protein sequences of repressor 1 in
L. reuteri.
Coding sequence
atgagcgtcacacctaacaaagtcttaattgatttgagacatgaaaaagg
tgaatctcaggctcaagcagctaagaatattggtatttcacaatcaatgc
tcgctatgcttgaagctggttatcgtaaaggcagcgatgacactaaaatc
aagattgctaactactacgataagtcagtggacactattttttttgcagt
taattgtcactcttag (SEQ ID NO: 1)
Protein sequence
MSVTPNKVLIDLRHEKGESQAQAAKNIGISQSMLAMLEAGYRKGSDDTKI
KIANYYDKSVDTIFFAVNCHS* (SEQ ID NO: 2)

TABLE 2
Coding and protein sequences of repressor 2 in
L. reuteri.
Coding sequence
atgccaggtactataaatcttaatttaattaaacaattacggtcaaaaaa
gggctttacgtatggtgatatggcttctgctttaggccttaaggaaccag
aaaaatattatcgtgtgaacaagggaaatatagatttcaagctaccgaat
taccgcctttagcaaagaaactagggatacctattgaaaaaatttttaaa
taa (SEQ ID NO: 3)
Protein sequence
MPGTINLNLIKQLRSKKGFTYGDMASALGLKEPEKYYRREQGKYRFQATE
LPPLAKKLGIPIEKIFK* (SEQ ID NO: 4)

TABLE 3
Coding and protein sequences of SucP in L. reuteri
sucP coding sequence
atgccaatcaaaaatgaagcaatgttaattacttactctgactcaatggg
taaaaatattaaagaaactcatgaagtattaaagaactatatcggtgatg
caatcggcggtgttcacttacttccattcttcccatcaactggtgaccgt
ggttttgcaccataccgttacgatgttgttgattctgcttttggtaactg
ggatgatgttgaagcattaggtgaagactactacttaatgtttgacttca
tgattaaccatatttccaagaagtctgaaatgtaccaagacttcaagaag
aagcacgatgattctaagtacaacgacttctttattcgttgggaaaagtt
ctgggaaaaagctggtaagaaccgtccaactcaagaagatgttgatttaa
tttacaagcgaaaggataaggctcctaagcaagaaattacctttgatgat
ggtactactgaaaacttatggaacactttcggtgaagaacaaattgatat
taatgttaagagtaaggtagctaacgaattcttcaaggaaacattaattg
acatggttaagcacggtgcagatatgattcgtcttgatgcctttgcttac
gctattaagaaggttggtactaatgatttcttcgttgaacctgaaatctg
ggatcttttaaatgaagttcaagatattttggctccttacaaggccatca
tccttcctgaaattcacgaacactacaccattccacaaaagatttcacaa
catgacttcttcatctatgactttaccttaccaatgactactctttatac
cctttactctggtaagactaaccgccttgctaagtggttaaagatgtcac
cgatgaagcaatttactactcttgatactcatgatggtattggggttgtt
gatgctaaggatatcttaactgatgatgaaatcgaatatgcatccaatga
attatacaaggttggtgctaacgttaagcggaaatactcaagtgctgaat
acaacaacttggatatttaccaaattaactctacttactactctgcatta
ggtgacgatgacaaagcttacttgctttctcgggcattccaagtatttgc
acctggtattccaatggtttactatgttggtttacttgctggttcaaatg
accttgaattacttgaaaagactaaggaaggtcggaacatcaaccgtcac
tactacactaaagaagaagttgcacaagaagttcaacgtccagtagttaa
gaatctcttagacttacttgcatggcggaataaatttgcagcctttgatc
ttgatggttcaattgaagtggaaacaccaactgaaacaactatcaaggtt
acgcggaaagataaggatggcaagaatgtggctgttcttgatgctgatgc
tgctaacaagactttcactattacagctaatggtgaaaaagtgatggaac
aaaaatag (SEQ ID NO: 5)
SucP protein sequence
MPIKNEAMLITYSDSMGKNIKETHEVLKNYIGDAIGGVHLLPFFPSTGDR
GFAPYRYDVVDSAFGNWDDVEALGEDYYLMFDFMINHISKKSEMYQDFKK
KHDDSKYNDFFIRWEKFWEKAGKNRPTQEDVDLIYKRKDKAPKQEITFDD
GTTENLWNTFGEEQIDINVKSKVANEFFKETLIDMVKHGADMIRLDAFAY
AIKKVGTNDFFVEPEIWDLLNEVQDILAPYKAIILPEIHEHYTIPQKISQ
HDFFIYDFTLPMTTLYTLYSGKTNRLAKWLKMSPMKQFTTLDTHDGIGVV
DAKDILTDDEIEYASNELYKVGANVKRKYSSAEYNNLDIYQINSTYYSAL
GDDDKAYLLSRAFQVFAPGIPMVYYVGLLAGSNDLELLEKTKEGRNINRH
YYTKEEVAQEVQRPVVKNLLDLLAWRNKFAAFDLDGSIEVETPTETTIKV
TRKDKDGKNVAVLDADAANKTFTITANGEKVMEQK* (SEQ ID NO:
6)

TABLE 4
Coding and protein sequences of AdhE in L. reuteri
adhE gene coding sequence
atgcctgctaacaacaagaaacaagttgaaaagaaagaattaactgctga
agaaaaaaagcaaaacgcccaaaagctagttgacgatttaatgactaaga
gtcaagctgcttttgaaaagttacgttactattcacaagaacaagttgac
aagatttgtcaggcaatggctctcgctgccgaagaacaccacatggactt
agctgttgatgctgctaacgaaactggtcgtggggttgctgaagataagg
ctatcaagaacatctacgcaagtgaatacatttggaacaacatccgtcac
gataagactgttggtattatcgaagacaatgatgaagaccaaactatcaa
aattgctgatccacttggtgtcattgccggaattgttccagttactaacc
ctacttcaacaacgatcttcaaatcaatcattagtgctaagacacggaat
acaatcatcttttctttccaccgtcaagcaatgaagtcatctatcaagac
tgcaaagattctccaagaagctgctgaaaaagccggtgcgccaaagaaca
tgattcaatggctccctgaaagtacccgcgaaaacactaccgcattactc
caacaccctaatactgctactattttagcaaccggtggtccttcattagt
taaggctgcctacagttctggtaaccctgctcttggtgttggtcctggta
acggtcctgcttacatcgaaaaaactgccaacatcgaacgttctgtttac
gacatcgttctttctaagacattcgataacggtatgatttgtgccactga
aaactcagttgttgttgatgaagaaatctacgacaaggttaaagaagaat
tccaaaagtggaactgttacttcttgaagccaaacgaaattgataaattt
actgatggctttattgacccagatcgtcatcaagttcgtggtccaatcgc
tggtcgttcagctaatgctattgctgacatgtgtggtattaaagtacctg
acaacactaaggttatcattgctgaatacgaaggtgttggtgacaagtac
ccactttcagctgaaaagctttcaccagtattaacaatgtacaaggcaac
ctctcacgaaaatgcctttgatatctgtgctcaattattacactacggtg
gtgaaggtcacactgctgctattcacacccttgatgatgatttagctact
aagtacggtcttgaaatgcgtgcttcacggatcattgttaactccccatc
tggtattggtggtattggtaacatctacaacaacatgactccatccctta
ctttaggtactggttcatacggtagtaactcaatttctcacaacgttact
gattgggacctcttaaacatcaaaacaattgcaaagcggcgtgaaaaccg
tcaatgggttaagattcccccaaaagtatactttcaacgcaactcactaa
aagaattgcaagatattccaaacattaaccgggcattcatcgttactggt
cctggaatgagcaagcgtggttacgttcaacgtgttatcgatcaattgcg
tcaacgccaaaacaacactgctttcttagtatttgatgacgttgaagaag
atccatcaacaaacactgttgaaaaaggtgttgccatgatgaatgacttc
aaacctgatacaattattgctcttggtggtggttcaccaatggatgctgc
taaggctatgtggatgttctatgagcacccagaaacttcatggtatgggg
ttatgcaaaagtaccttgatattcggaagcgtgcttaccaaatcaagaag
cctactaagtctcaacttattggtatccctactacatcaggtactggttc
agaagttactccatttgcggttattaccgattcaaaaactcatgttaagt
acccacttgctgactacgccttaacaccaaacattgcaatcgttgactca
caattcgttgaaactgttccagcaaaaactactgcttggactggactaga
tgttttatgtcacgctactgaatcatatgtttctgttatggcaactgact
acactcgtggttggtcactacaaaccatcaagggtgttatggaaaacctt
cctaagtcagttcaaggtgataagttagctcgtcgtaagatgcacgactt
ctcaacaatggccgggatggcatttggtcaagccttcttaggaattaacc
actcccttgcccacaagatgggtggagcattcggtcttcctcacggtttg
cttatcgctattgcaatgccacaagtaattcgctttaacgcaaaacgtcc
acaaaagcttgctctctggcctcactatgagacttaccatgcaactaagg
actacgctgacattgcacggttcattggtttgaaaggcaacactgatgaa
gaattagctgaagcatatgctaagaaagttatcgaacttgctcacgaatg
tggtgttaagcttagtcttaaggacaatggtgttacacgtgaagaatttg
ataaggcggttgacgatcttgctcgcttagcttacgaagatcaatgtact
actactaacccagttgaaccacttgttagccaactcaaggaattacttga
acgttgctacgatggtactggcgttgaagaaaaataa (SEQ ID NO:
7)
AdhE protein sequence
MPANNKKQVEKKELTAEEKKQNAQKLVDDLMTKSQAAFEKLRYYSQEQVD
KICQAMALAAEEHHMDLAVDAANETGRGVAEDKAIKNIYASEYIWNNIRH
DKTVGIIEDNDEDQTIKIADPLGVIAGIVPVTNPTSTTIFKSIISAKTRN
TIIFSFHRQAMKSSIKTAKILQEAAEKAGAPKNMIQWLPESTRENTTALL
QHPNTATILATGGPSLVKAAYSSGNPALGVGPGNGPAYIEKTANIERSVY
DIVLSKTFDNGMICATENSVVVDEEIYDKVKEEFQKWNCYFLKPNEIDKF
TDGFIDPDRHQVRGPIAGRSANAIADMCGIKVPDNTKVIIAEYEGVGDKY
PLSAEKLSPVLTMYKATSHENAFDICAQLLHYGGEGHTAAIHTLDDDLAT
KYGLEMRASRIIVNSPSGIGGIGNIYNNMTPSLTLGTGSYGSNSISHNVT
DWDLLNIKTIAKRRENRQWVKIPPKVYFQRNSLKELQDIPNINRAFIVTG
PGMSKRGYVQRVIDQLRQRQNNTAFLVFDDVEEDPSTNTVEKGVAMMNDF
KPDTIIALGGGSPMDAAKAMWMFYEHPETSWYGVMQKYLDIRKRAYQIKK
PTKSQLIGIPTTSGTGSEVTPFAVITDSKTHVKYPLADYALTPNIAIVDS
QFVETVPAKTTAWTGLDVLCHATESYVSVMATDYTRGWSLQTIKGVMENL
PKSVQGDKLARRKMHDFSTMAGMAFGQAFLGINHSLAHKMGGAFGLPHGL
LIAIAMPQVIRFNAKRPQKLALWPHYETYHATKDYADIARFIGLKGNTDE
ELAEAYAKKVIELAHECGVKLSLKDNGVTREEFDKAVDDLARLAYEDQCT
TTNPVEPLVSQLKELLERCYDGTGVEEK* (SEQ ID NO: 8)

TABLE 5
DNA fragment and primers used in this study.

| Name | Sequence (5′→3′) |
|---|---|
| eftu-R | Cgaattaatagaaaaacattagtcaaatacatttaca |
| | aatgaacagatagttgatattatatttaagaattctt |
| | cttcagagtctaagattaaagctttcaattggcgaaa |
| | agaagttgtacaatatgtataagggtatgtcagtcac |
| | cgaatcagatgatctggcattatacttgtaaattatc |
| | aggaggttttcattaatgtctgtaactccaaataaag |
| | ttttaattgatttgcggcatgaaaagggagaatcaca |
| | agctcaggctgctaagaatattggtatttcacaatcg |
| | atgctcgcaatgttagaagctggttatcgaaagggtt |
| | ctgatgatacaaagattaaaatcgctaattactacga |
| | caaatctgttgacactatctttttcgccgttaattgt |
| | cattcctag (SEQ ID NO: 9) |
| Backbone-F | gagtctagattagctgtcagcttca (SEQ ID NO: |
| | 10) |
| Backbone-R | gaggaattcggtaccccgggttcga (SEQ ID NO: |
| | 11) |
| Bridging | gccgatgaagctgacagctaatctagactccgaatta |
| oligo 1 | atagaaaaacattagtcaaatac (SEQ ID NO: |
| | 12) |
| Bridging | atctttttcgccgttaattgtcattcctaggaggaat |
| oligo 2 | tcggtaccccgggttcgaaggcg (SEQ ID NO: |
| | 13) |
| Screening- | aaggcaaagaaaccagtgaagaagt (SEQ ID NO: |
| F | 14) |
| Screening- | cgctttgattgttctatcgaaagc (SEQ ID NO: |
| R | 15) |

1. A system comprising a microbial host cell and a recombinant plasmid capable of replicating in the host cell, wherein the plasmid comprises one or more plasmid-maintenance genes configured to express in the host cell and a gene of interest configured to express in the host cell, wherein the one or more plasmid-maintenance genes comprise one or more of a prophage repressor gene, a sucrose phosphorylase gene, and an alcohol/aldehyde dehydrogenase gene.

2. The system of claim 1, wherein the one or more plasmid-maintenance genes comprises a prophage repressor gene.

3. The system of claim 2, wherein the host cell comprises a genome comprising a prophage.

4. The system of claim 3, wherein the prophage repressor gene encodes a repressor of the prophage.

5. The system of claim 2, wherein the prophage repressor gene encodes a protein comprising a sequence at least 80% identical to a sequence selected from the group consisting of SEQ ID NOS:2 and 4.

6. The system of claim 1, wherein the one or more plasmid maintenance genes comprises a sucrose phosphorylase gene.

7. The system of claim 6, wherein the sucrose phosphorylase gene encodes a protein comprising a sequence at least 80% identical to SEQ ID NO:6.

8. The system of claim 6, wherein the host cell comprises a mutation that reduces sucrose phosphorylase activity with respect to a native form of the host cell.

9. The system of claim 6, wherein the host cell comprises an inactivating mutation to a native sucrose phosphorylase gene present in a native form of the host cell.

10. The system of claim 9, wherein the native sucrose phosphorylase gene encodes a protein comprising a sequence at least 80% identical to SEQ ID NO:6.

11. The system of claim 1, wherein the one or more plasmid maintenance genes comprises an alcohol/aldehyde dehydrogenase gene.

12. The system of claim 11, wherein the alcohol/aldehyde dehydrogenase gene encodes a protein comprising a sequence at least 80% identical to SEQ ID NO:8.

13. The system of claim 11, wherein the host cell comprises a mutation that reduces alcohol/aldehyde dehydrogenase activity with respect to a native form of the host cell.

14. The system of claim 11, wherein the host cell comprises an inactivating mutation to a native alcohol/aldehyde dehydrogenase gene present in a native form of the host cell.

15. The system of claim 14, wherein the native sucrose phosphorylase gene encodes a protein comprising a sequence at least 80% identical to SEQ ID NO:8.

16. The system of claim 1, wherein the plasmid is devoid of any antibiotic resistance gene.

17. The system of claim 1, wherein the host cell is a bacterium.

18. The system of claim 1, wherein the host cell is a member of Limosilactobacillus.

19. The system of claim 1, wherein the host cell is Limosilactobacillus reuteri.

20. A method of using the system of claim 1, comprising growing the host cell with the plasmid comprised within the host cell.

21-29. (canceled)


SEQUENCE LISTING (15 sequences)

SEQ ID NO: 1 - DNA, 216 bp, Limosilactobacillus reuteri
  source 1..216: mol_type = genomic DNA
atgagcgtcacacctaacaaagtcttaattgatttgagacatgaaaaaggtgaatctcaggctcaagcagctaagaatattggtatttcacaatcaatgctcgctatgcttgaagctggttatcgtaaaggcagcgatgacactaaaatcaagattgctaactactacgataagtcagtggacactattttttttgcagttaattgtcactcttag

SEQ ID NO: 2 - protein, 71 aa, Limosilactobacillus reuteri
MSVTPNKVLIDLRHEKGESQAQAAKNIGISQSMLAMLEAGYRKGSDDTKIKIANYYDKSVDTIFFAVNCHS

SEQ ID NO: 3 - DNA, 204 bp, Limosilactobacillus reuteri
  source 1..204: mol_type = genomic DNA
atgccaggtactataaatcttaatttaattaaacaattacggtcaaaaaagggctttacgtatggtgatatggcttctgctttaggccttaaggaaccagaaaaatattatcgtcgtgaacaagggaaatatagatttcaagctaccgaattaccgcctttagcaaagaaactagggatacctattgaaaaaatttttaaataa

SEQ ID NO: 4 - protein, 67 aa, Limosilactobacillus reuteri
MPGTINLNLIKQLRSKKGFTYGDMASALGLKEPEKYYRREQGKYRFQATELPPLAKKLGIPIEKIFK

SEQ ID NO: 5 - DNA, 1458 bp, Limosilactobacillus reuteri
  source 1..1458: mol_type = genomic DNA
atgccaatcaaaaatgaagcaatgttaattacttactctgactcaatgggtaaaaatattaaagaaactcatgaagtattaaagaactatatcggtgatgcaatcggcggtgttcacttacttccattcttcccatcaactggtgaccgtggttttgcaccataccgttacgatgttgttgattctgcttttggtaactgggatgatgttgaagcattaggtgaagactactacttaatgtttgacttcatgattaaccatatttccaagaagtctgaaatgtaccaagacttcaagaagaagcacgatgattctaagtacaacgacttctttattcgttgggaaaagttctgggaaaaagctggtaagaaccgtccaactcaagaagatgttgatttaatttacaagcgaaaggataaggctcctaagcaagaaattacctttgatgatggtactactgaaaacttatggaacactttcggtgaagaacaaattgatattaatgttaagagtaaggtagctaacgaattcttcaaggaaacattaattgacatggttaagcacggtgcagatatgattcgtcttgatgcctttgcttacgctattaagaaggttggtactaatgatttcttcgttgaacctgaaatctgggatcttttaaatgaagttcaagatattttggctccttacaaggccatcatccttcctgaaattcacgaacactacaccattccacaaaagatttcacaacatgacttcttcatctatgactttaccttaccaatgactactctttataccctttactctggtaagactaaccgccttgctaagtggttaaagatgtcaccgatgaagcaatttactactcttgatactcatgatggtattggggttgttgatgctaaggatatcttaactgatgatgaaatcgaatatgcatccaatgaattatacaaggttggtgctaacgttaagcggaaatactcaagtgctgaatacaacaacttggatatttaccaaattaactctacttactactctgcattaggtgacgatgacaaagcttacttgctttctcgggcattccaagtatttgcacctggtattccaatggtttactatgttggtttacttgctggttcaaatgaccttgaattacttgaaaagactaaggaaggtcggaacatcaaccgtcactactacactaaagaagaagttgcacaagaagttcaacgtccagtagttaagaatctcttagacttacttgcatggcggaataaatttgcagcctttgatcttgatggttcaattgaagtggaaacaccaactgaaacaactatcaaggttacgcggaaagataaggatggcaagaatgtggctgttcttgatgctgatgctgctaacaagactttcactattacagctaatggtgaaaaagtgatggaacaaaaatag

SEQ ID NO: 6 - protein, 485 aa, Limosilactobacillus reuteri
MPIKNEAMLITYSDSMGKNIKETHEVLKNYIGDAIGGVHLLPFFPSTGDRGFAPYRYDVVDSAFGNWDDVEALGEDYYLMFDFMINHISKKSEMYQDFKKKHDDSKYNDFFIRWEKFWEKAGKNRPTQEDVDLIYKRKDKAPKQEITFDDGTTENLWNTFGEEQIDINVKSKVANEFFKETLIDMVKHGADMIRLDAFAYAIKKVGTNDFFVEPEIWDLLNEVQDILAPYKAIILPEIHEHYTIPQKISQHDFFIYDFTLPMTTLYTLYSGKTNRLAKWLKMSPMKQFTTLDTHDGIGVVDAKDILTDDEIEYASNELYKVGANVKRKYSSAEYNNLDIYQINSTYYSALGDDDKAYLLSRAFQVFAPGIPMVYYVGLLAGSNDLELLEKTKEGRNINRHYYTKEEVAQEVQRPVVKNLLDLLAWRNKFAAFDLDGSIEVETPTETTIKVTRKDKDGKNVAVLDADAANKTFTITANGEKVMEQK

SEQ ID NO: 7 - DNA, 2637 bp, Limosilactobacillus reuteri
  source 1..2637: mol_type = genomic DNA
atgcctgctaacaacaagaaacaagttgaaaagaaagaattaactgctgaagaaaaaaagcaaaacgcccaaaagctagttgacgatttaatgactaagagtcaagctgcttttgaaaagttacgttactattcacaagaacaagttgacaagatttgtcaggcaatggctctcgctgccgaagaacaccacatggacttagctgttgatgctgctaacgaaactggtcgtggggttgctgaagataaggctatcaagaacatctacgcaagtgaatacatttggaacaacatccgtcacgataagactgttggtattatcgaagacaatgatgaagaccaaactatcaaaattgctgatccacttggtgtcattgccggaattgttccagttactaaccctacttcaacaacgatcttcaaatcaatcattagtgctaagacacggaatacaatcatcttttctttccaccgtcaagcaatgaagtcatctatcaagactgcaaagattctccaagaagctgctgaaaaagccggtgcgccaaagaacatgattcaatggctccctgaaagtacccgcgaaaacactaccgcattactccaacaccctaatactgctactattttagcaaccggtggtccttcattagttaaggctgcctacagttctggtaaccctgctcttggtgttggtcctggtaacggtcctgcttacatcgaaaaaactgccaacatcgaacgttctgtttacgacatcgttctttctaagacattcgataacggtatgatttgtgccactgaaaactcagttgttgttgatgaagaaatctacgacaaggttaaagaagaattccaaaagtggaactgttacttcttgaagccaaacgaaattgataaatttactgatggctttattgacccagatcgtcatcaagttcgtggtccaatcgctggtcgttcagctaatgctattgctgacatgtgtggtattaaagtacctgacaacactaaggttatcattgctgaatacgaaggtgttggtgacaagtacccactttcagctgaaaagctttcaccagtattaacaatgtacaaggcaacctctcacgaaaatgcctttgatatctgtgctcaattattacactacggtggtgaaggtcacactgctgctattcacacccttgatgatgatttagctactaagtacggtcttgaaatgcgtgcttcacggatcattgttaactccccatctggtattggtggtattggtaacatctacaacaacatgactccatcccttactttaggtactggttcatacggtagtaactcaatttctcacaacgttactgattgggacctcttaaacatcaaaacaattgcaaagcggcgtgaaaaccgtcaatgggttaagattcccccaaaagtatactttcaacgcaactcactaaaagaattgcaagatattccaaacattaaccgggcattcatcgttactggtcctggaatgagcaagcgtggttacgttcaacgtgttatcgatcaattgcgtcaacgccaaaacaacactgctttcttagtatttgatgacgttgaagaagatccatcaacaaacactgttgaaaaaggtgttgccatgatgaatgacttcaaacctgatacaattattgctcttggtggtggttcaccaatggatgctgctaaggctatgtggatgttctatgagcacccagaaacttcatggtatggggttatgcaaaagtaccttgatattcggaagcgtgcttaccaaatcaagaagcctactaagtctcaacttattggtatccctactacatcaggtactggttcagaagttactccatttgcggttattaccgattcaaaaactcatgttaagtacccacttgctgactacgccttaacaccaaacattgcaatcgttgactcacaattcgttgaaactgttccagcaaaaactactgcttggactggactagatgttttatgtcacgctactgaatcatatgtttctgttatggcaactgactacactcgtggttggtcactacaaaccatcaagggtgttatggaaaaccttcctaagtcagttcaaggtgataagttagctcgtcgtaagatgcacgacttctcaacaatggccgggatggcatttggtcaagccttcttaggaattaaccactcccttgcccacaagatgggtggagcattcggtcttcctcacggtttgcttatcgctattgcaatgccacaagtaattcgctttaacgcaaaacgtccacaaaagcttgctctctggcctcactatgagacttaccatgcaactaaggactacgctgacattgcacggttcattggtttgaaaggcaacactgatgaagaattagctgaagcatatgctaagaaagttatcgaacttgctcacgaatgtggtgttaagcttagtcttaaggacaatggtgttacacgtgaagaatttgataaggcggttgacgatcttgctcgcttagcttacgaagatcaatgtactactactaacccagttgaaccacttgttagccaactcaaggaattacttgaacgttgctacgatggtactggcgttgaagaaaaataa

SEQ ID NO: 8 - protein, 878 aa, Limosilactobacillus reuteri
MPANNKKQVEKKELTAEEKKQNAQKLVDDLMTKSQAAFEKLRYYSQEQVDKICQAMALAAEEHHMDLAVDAANETGRGVAEDKAIKNIYASEYIWNNIRHDKTVGIIEDNDEDQTIKIADPLGVIAGIVPVTNPTSTTIFKSIISAKTRNTIIFSFHRQAMKSSIKTAKILQEAAEKAGAPKNMIQWLPESTRENTTALLQHPNTATILATGGPSLVKAAYSSGNPALGVGPGNGPAYIEKTANIERSVYDIVLSKTFDNGMICATENSVVVDEEIYDKVKEEFQKWNCYFLKPNEIDKFTDGFIDPDRHQVRGPIAGRSANAIADMCGIKVPDNTKVIIAEYEGVGDKYPLSAEKLSPVLTMYKATSHENAFDICAQLLHYGGEGHTAAIHTLDDDLATKYGLEMRASRIIVNSPSGIGGIGNIYNNMTPSLTLGTGSYGSNSISHNVTDWDLLNIKTIAKRRENRQWVKIPPKVYFQRNSLKELQDIPNINRAFIVTGPGMSKRGYVQRVIDQLRQRQNNTAFLVFDDVEEDPSTNTVEKGVAMMNDFKPDTIIALGGGSPMDAAKAMWMFYEHPETSWYGVMQKYLDIRKRAYQIKKPTKSQLIGIPTTSGTGSEVTPFAVITDSKTHVKYPLADYALTPNIAIVDSQFVETVPAKTTAWTGLDVLCHATESYVSVMATDYTRGWSLQTIKGVMENLPKSVQGDKLARRKMHDFSTMAGMAFGQAFLGINHSLAHKMGGAFGLPHGLLIAIAMPQVIRFNAKRPQKLALWPHYETYHATKDYADIARFIGLKGNTDEELAEAYAKKVIELAHECGVKLSLKDNGVTREEFDKAVDDLARLAYEDQCTTTNPVEPLVSQLKELLERCYDGTGVEEK

SEQ ID NO: 9 - DNA, 416 bp, synthetic construct
  source 1..416: mol_type = other DNA
cgaattaatagaaaaacattagtcaaatacatttacaaatgaacagatagttgatattatatttaagaattcttcttcagagtctaagattaaagctttcaattggcgaaaagaagttgtacaatatgtataagggtatgtcagtcaccgaatcagatgatctggcattatacttgtaaattatcaggaggttttcattaatgtctgtaactccaaataaagttttaattgatttgcggcatgaaaagggagaatcacaagctcaggctgctaagaatattggtatttcacaatcgatgctcgcaatgttagaagctggttatcgaaagggttctgatgatacaaagattaaaatcgctaattactacgacaaatctgttgacactatctttttcgccgttaattgtcattcctag

SEQ ID NO: 10 - DNA, 25 bp, synthetic construct
  source 1..25: mol_type = other DNA
gagtctagattagctgtcagcttca

SEQ ID NO: 11 - DNA, 25 bp, synthetic construct
  source 1..25: mol_type = other DNA
gaggaattcggtaccccgggttcga

SEQ ID NO: 12 - DNA, 60 bp, synthetic construct
  source 1..60: mol_type = other DNA
gccgatgaagctgacagctaatctagactccgaattaatagaaaaacattagtcaaatac

SEQ ID NO: 13 - DNA, 60 bp, synthetic construct
  source 1..60: mol_type = other DNA
atctttttcgccgttaattgtcattcctaggaggaattcggtaccccgggttcgaaggcg

SEQ ID NO: 14 - DNA, 25 bp, synthetic construct
  source 1..25: mol_type = other DNA
aaggcaaagaaaccagtgaagaagt

SEQ ID NO: 15 - DNA, 24 bp, synthetic construct
  source 1..24: mol_type = other DNA
cgctttgattgttctatcgaaagc